(19) United States
(12) Patent Application Publication
(10) Pub. No.: US 2004/0109797 A1
(43) Pub. Date: Jun. 10, 2004

(54) Ozone destructor

(76) Inventor: Coby Grove, (US)

Correspondence Address: PERKINS COIE LLP, POST OFFICE BOX 1208, SEATTLE, WA, 98111-1208, US

(21) Appl. No.: 10/315,609

(22) Filed: Dec. 10, 2002

(51) Int. Cl. A61L 9/00; A61L 2/00; A62B 7/08; B01J 19/08

(52) U.S. Cl. 422/186.07; 422/1; 422/4; 422/5; 422/22; 422/28; 422/121; 422/123; 422/292; 422/305

(57) ABSTRACT

A system for processing a wafer using ozone includes an ozone gas source connecting into the process chamber. Wafers are held within a holder or fixture in the chamber. A chamber exhaust line connects the process chamber to an inlet at the top end of an ozone destructor. A system or cabinet exhaust line extends from an outlet also at the top end of the ozone destructor. A canister within the ozone destructor contains a catalyst. Exhaust flow from the process chamber moves down through the ozone destructor and then up through the catalyst. Saturation of the catalyst by condensing water vapor and loss of catalytic efficiency, is reduced. As the process chamber and chamber exhaust line are better isolated from the catalyst, potential for catalyst particles moving into the process chamber or chamber exhaust line, or for condensing vapor to back up in the chamber exhaust line, are reduced.

### FIELD OF THE INVENTION

The field of the invention is processing of workpieces, such as semiconductor wafers, with ozone.

### BACKGROUND OF THE INVENTION

Semiconductor devices, such as micro-processors, memory chips, and various other electronic devices are manufactured using well-known techniques. These types of electronic devices are typically manufactured from silicon or gallium arsenide wafers, although other materials may be considered as well. Additional electronic products, such as flat panel displays and other optical media, rigid disk memories, thin film head device substrates, compact disk substrates, MEMS devices, and similar flat media are manufactured using similar techniques using both semiconductor and non-semiconductor materials. These workpieces are collectively referred to here as wafers or articles.

In manufacturing articles, the base materials, such as silicon wafers, are often exposed to various chemicals, to add or remove layers of material; to change the characteristics of the base material, for cleaning; or for other purposes. These processing steps are often carried out by spinning the wafers within a processing chamber. Liquid and gas phase chemicals are sprayed onto, or otherwise applied to, the wafers in the processing chamber. After a predetermined time, the liquid processing chemicals are drawn off and out of the chamber, usually at the bottom of the chamber, while the gases and/or vapors, are separately drawn or pumped out of the chamber and then vented to the outside.

For certain processing steps, ozone is introduced into the processing chamber. Ozone is typically introduced with water, sometimes containing dilute amounts of chemical. The ozone is then removed from the processing chamber (optionally along with other chemicals, such as acid vapors) and then exhausted to the atmosphere.

However, ozone is a highly chemically reactive gas. In high concentrations, it can become toxic to humans. The exhaust mixture of ozone can therefore be both toxic, and highly corrosive. Consequently, handling the gas exhaust from a processing chamber generally requires special procedures. For example, components such as ducts, etc. generally must be made of PVDF or other plastics which resist corrosion. Leak detectors may also be employed to detect any leaks in the pipes or ducts carrying the exhaust gases from the processing chamber to the outside.

Ozone converters or destructors have been used to convert ozone in an exhaust flow into oxygen. These converters or destructors typically use catalysts, such as maganese dioxide. The catalyst, however, lose efficiency or ability to catalyze, when they become saturated with condensation. It is also important to prevent stray catalyst particles from moving into the processing chamber, where they can cause contamination.

Accordingly, it is a further object of the invention to provide an improved ozone destructor for destroying ozone, by converting ozone into oxygen.

It is also an object of the invention to provide an improved apparatus and method for processing articles using ozone, and an improved ozone destructor.

### SUMMARY OF THE INVENTION

In a first aspect of the invention, a system for processing a wafer or article includes an ozone gas source connecting to a process chamber. One or more wafers are held within a holder or fixture within the process chamber. A chamber exhaust line or pipe connects the process chamber to the inlet adjacent an upper end of an ozone destructor. A system, cabinet, or enclosure exhaust line connects with an outlet of the ozone destructor adjacent to an upper end of the ozone destructor. Gases and/or vapors exhausted from the process chamber move vertically upwardly through a catalyst in the ozone destructor. As the inlet of the ozone destructor is vertically above the catalyst, movement of catalyst particles into the chamber exhaust line, or the process chamber, is reduced or eliminated. In addition, the potential for liquid within the ozone destructor, resulting from condensation of exhaust vapors, moving back into the chamber exhaust line or process chamber is also reduced or eliminated.

In a second aspect of the invention, in a system and method for processing an article or wafer, exhaust gases and/or vapors move upwardly through a catalyst. Ozone in the exhaust flow is converted to oxygen. Saturation of the catalyst with condensation is reduced, as liquid condensation within the catalyst drains downwardly, via gravity, out of the catalyst or catalyst canister. As saturation of the catalyst (e.g., with condensed water vapor) is reduced, the catalyst maintains efficiency in converting ozone to oxygen.

In a third aspect of the invention, a device for destroying or converting ozone includes a container containing a catalyst and forming a flow path through the catalyst. The flow path has a first flow segment extending in a down direction, towards a lower end of the container. A second flow segment connects with the first segment. The second segment extends in an upward direction through the catalyst, and towards an upper end of the container. Saturation of the catalyst with liquid from condensed vapors is reduced, as any condensation within the catalyst can freely drain out. This helps to prevent reduced catalytic performance caused by saturation of the catalyst. Potential contamination caused by inadvertent migration or movement of catalyst particles is also reduced. The added complexity, cost, space requirements, and other disadvantages of using a demister or heater, to maintain catalyst performance, is avoided.

Each of the above described aspects helps to improve wafer manufacturing. The invention resides as well in subsystems, components, and method steps as described below. The invention is also friendly to the environment by reducing or preventing release of ozone gas into the environment.

### BRIEF DESCRIPTION OF THE DRAWINGS

Additional aspects and advantages of the invention will become apparent from the following detailed description and drawings. In the drawings, the same element number indicates the same element in each of the views.

FIG. 1 is a perspective view of an automated wafer or article processing system. In this automated system, wafers are loaded and unloaded from process chambers by a robot.

FIG. 2 is a perspective view of a manual or non-automated processing system.

FIG. 3 is an exploded perspective view of the ozone destructor shown in FIGS. 1 and 2.

FIG. 4 is a section view of the ozone destructor shown in FIGS. 1-3.

### DETAIL DESCRIPTION OF THE DRAWINGS

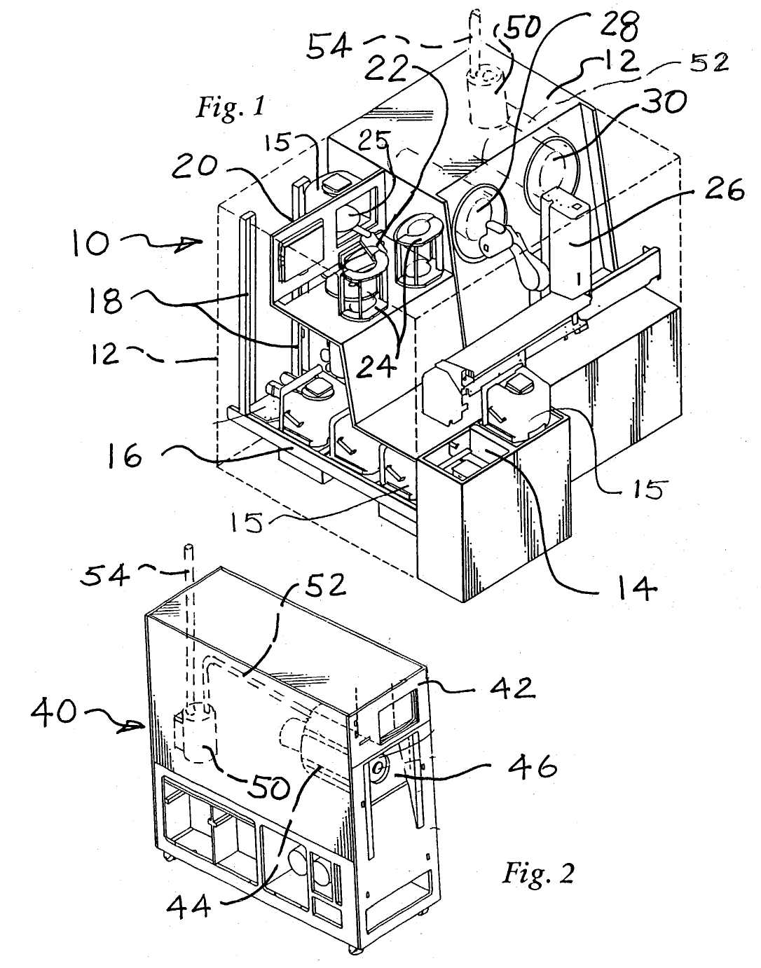

Turning now in detail to the drawings, as shown in FIG. 1, an automated processing system 10 has an indexer 16, elevators 18, and docking station 20, within an enclosure 12. A load/unload unit 14 moves containers or boxes 15 into and out of the enclosure 12. The wafers, articles, or workpieces 25 are contained within the boxes 15. The indexer 16 holds or stores boxes 15 within the enclosure 12 and moves the boxes 15 rearwardly towards an elevator 18. The elevator 18 lifts a box to the docking station 20. The docking station robot 22 moves wafers 25 from a box 15 at the docking station 20 into a carrier 24. A process robot 26 moves the carrier 24 holding the wafers 25 to a first process chamber 28 or a second process chamber 30. Sources of process liquids, vapors, or gases connect with the process chambers 28 and 30, including sources of ozone gas, and optionally including deionized water as well as process liquids or gases, such as acids, solvents, surfactants, air, or nitrogen. Additional description of the automated processing system 10 shown in FIG. 1 is in U.S. patent application Ser. Nos. 09/612,009 and 09/735,154, both incorporated herein by reference.

FIG. 2 shows a non-automated processing system 40 having a process chamber 44 within an enclosure 42. Wafers 25 are loaded into the process chamber 44 manually, via a chamber door 46.

In both processing systems 10 and 40, a chamber exhaust line, pipe or duct 52 extends between or connects the process chamber 28, 30, 44 with an ozone destructor 50. A system or enclosure exhaust line 54 extends from the ozone destructor 50 out of the enclosure 12 or 42, typically to a facility exhaust line. While the systems 10 and 40 are intended for batch processing, the ozone destructor 50 may be used in any system, manual or automated, batch or single wafer processor, that uses ozone.

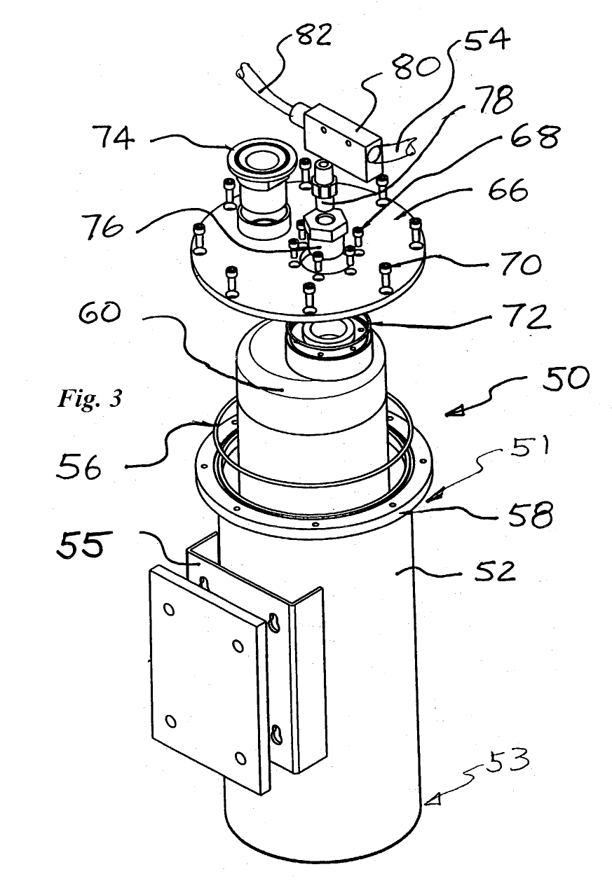

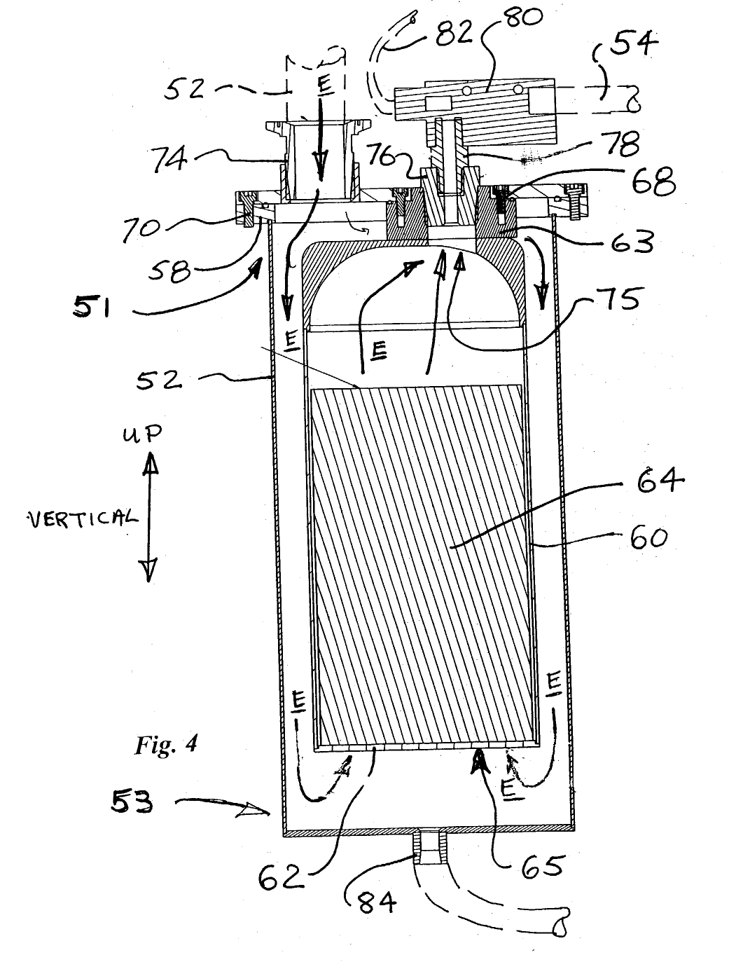

Referring to FIGS. 3 and 4, the ozone destructor 50 has an upper end 51 and a lower end 53. The exhaust line 52 from the process chamber 28, 30, or 44 connects at an inlet 74 at or near the upper end 51 of the ozone destructor 50. As shown with the arrows E in FIG. 4, exhaust from the process chamber flows down within the ozone destructor 50, reverses direction, and then flows up through a catalyst 64 and then out of the ozone destructor 50 through an outlet to the system or enclosure exhaust line 54. The drain line is either valved or trapped to prevent ozone flow to the drain.

While FIG. 4 shows the ozone destructor 50 in a vertically upright position, with exhaust gas flowing vertically up through the catalyst 64, the ozone destructor 50 may also have a different position or orientation, so long as there is a vertical component to the flow direction through the catalyst.

Referring still to FIG. 4, by having the exhaust gas enter and exit at or adjacent to the top of the ozone destructor 50, and flow upwardly through the catalyst 64, the catalyst 64 is better isolated from the chamber exhaust line 52 and the process chambers. Consequently, potential for catalyst particles to move into the chamber exhaust line or process chambers is reduced, as catalyst particles would have to move against both gravity and exhaust flow to move into the chamber exhaust line 52. As exhaust also flows up through the catalyst, any condensation within the catalyst can run down, via gravity and drain out. This helps to prevent loss of catalytic action due to saturation of the catalyst. Ozone in exhaust flow moving through the catalyst 64 is converted into oxygen, which can then be vented out through the system exhaust 54.

Referring still to FIGS. 3 and 4, the following additional detailed description relates to a preferred ozone destructor design, without individually describing any single essential elements of the invention. The ozone destructor 50 has an outer container 52 which may be supported on or in an enclosure with a mounting bracket 55. A lid 66 is supported and attached to a flange 58 of the outer container 52. An o-ring or seal 56 seals the lid 66 to the outer container 52. Lid bolts or fasteners 70 secure the lid 66 to the outer container 52. An inner container or canister 60 contains the catalyst 64, typically a manganese dioxide-based catalyst suitable for decomposing ozone into oxygen. A perforated plate 62 at the lower end of the canister 60 holds the catalyst 64 (typically beads of solid material) within the canister 60. The perforated plate 62 has openings allowing exhaust gas to enter at the bottom, with the openings forming canister inlets 65. A canister collar 63 is attached at the top or upper end of the canister or inner container 60. Canister mounting bolts 68 attach the canister 60 via the canister collar 63 to the lid 66. A canister o-ring 72 seals the canister collar 63 against the lid 66. A lid bushing 76 extends through an opening in the lid and into the canister collar 63, connecting with the canister outlet 75, at the top or upper end of the canister 60. A pipe nipple 78 connects an aspirator 80 with the canister outlet 75 through the lid bushing 76. A clean dry air supply line 82 connects to the inlet side of the aspirator 80. The outlet side of the aspirator 80 connects, directly or indirectly to the system exhaust line 54. The aspirator 80 may be replaced by an air amplifier. The inlet 74 provides an entry flow path into the ozone destructor 50, through the lid 66.

Referring to FIG. 4, the canister or inner container 60 containing the catalyst 64 is preferably suspended within the outer container 52. As shown in FIG. 3, the outer container 52 and inner container 60 are preferably cylindrical, although other cross section shapes may also be used. Referring to FIG. 4, an annular flow path extends from the inlet 74 down within the ozone destructor 50, between the outside walls of the inner container 60 and the inside walls of the outer container 52 to the canister inlets 65.

In use within the system 10 or 40, wafers 25 are processed within the process chambers 28, 30, 44. Ozone gas is introduced into a process chamber, to perform a desired process step on the wafers 25, for example, in a step for removing organic contaminants from the wafers 25. Gases and/or vapors are removed from the process chambers through the chamber exhaust line 52 which connects to the inlet or inlet fittings 74 of the ozone destructor 50.

Referring to FIG. 4, the exhaust gas moves into the ozone destructor at or near the upper end 51 and moves downwardly, as shown by the arrows E. The exhaust flow E moves to the lower end 53 of the ozone destructor 50, reverses direction, and flows upwardly through the perforated plate 62 and into the canister 60. As the exhaust flows through the catalyst 64 within the canister 60, ozone within the exhaust gas E is converted into oxygen. The aspirator or air amplifier 80 helps to draw exhaust gas flow through the ozone destructor 50, thereby eliminating any back pressure on the process chamber. Oxygen converted by the catalyst 64, and any other exhaust gas components, move upwardly through the canister outlet 75, through the aspirator 80, and out via the system exhaust line 54.

If water vapor condenses within the catalyst 64, the liquid water drains out downwardly through the perforated plate 62 and collects at the lower end 53 of the outer container 52, where it can be removed by the liquid drain line 84. Consequently, saturation of the catalyst 64 with condensation is avoided. As the exhaust flow inlet into the ozone destructor 50 is at or near the upper end 51, the potential for catalyst particles getting into the chamber exhaust line 52 or the process chambers is greatly reduced. Similarly, the potential for condensed liquid to block the exhaust line 52 is reduced or eliminated.

Preferably, the components of the ozone destructor 50 coming in contact with exhaust gas are made of stainless steel having a PFA coating to better resist corrosion by chemical vapors in the exhaust flow.

Thus, a novel processing system and ozone destructor have been shown and described. Various changes may be made without departing from the spirit and scope of the invention. The invention, therefore, should not be limited, except by the following claims, and their equivalents.

1. A system for processing an article comprising:
a process chamber;
an ozone gas source connecting into the process chamber;
a holder in the process chamber for holding at least one article;
an ozone destructor having an upper end, and an inlet and an outlet adjacent to the upper end;
a chamber exhaust line connecting from the process chamber to the inlet of the ozone destructor; and
a system exhaust line connecting to the outlet of the ozone destructor.

2. The system of claim 1 further comprising an enclosure, with the process chamber and the ozone destructor within the enclosure.

3. The system of claim 1 with the ozone destructor further including a canister supported within an outer container, and with the canister holding a catalyst.

4. The system of claim 3 wherein the canister is suspended from a lid on the outer container.

5. The system of claim 4 wherein the ozone destructor has a gas flow path extending from the inlet adjacent to the upper end of the destructor, through at least one opening into the canister adjacent to a lower end of the ozone destructor, upwardly through the catalyst in the canister, and then through the outlet adjacent to the upper end of the ozone destructor.

6. The system of claim 3 further comprising an aspirator or an air amplifier connecting with the outlet, for drawing ozone gas through the catalyst.

7. The system of claim 3 further comprising a liquid drain at the lower end of the ozone destructor.

8. The system of claim 3 further comprising a perforated plate at the lower end of the canister, for allowing ozone to enter at the lower end of the canister.

9. The system of claim 1 wherein the holder comprises a rotor for rotating the article within the chamber.

10. A device for destroying ozone, comprising:
a container having an upper end and a lower end;
a container inlet in the container, adjacent to the upper end of the container;
a container outlet in the container, adjacent to the upper end of the container;
a canister within the container, with the canister having an upper end and a lower end;
a canister inlet adjacent to the lower end of the canister;
a canister outlet adjacent to the upper end of the canister, with the canister outlet leading into the container outlet; and
a catalyst within the canister, above the canister inlet and below the canister outlet.

11. The device of claim 10 wherein the canister inlet faces downwardly.

12. The device of claim 10 further comprising a liquid drain opening adjacent to the lower end of the container.

13. The device of claim 10 further comprising a lid at the upper end of the container, with the upper end of the canister attached to the lid, to suspend the canister within the container.

14. The device of claim 10 wherein the lower end of the canister is spaced apart from the lower end of the container.

15. The device of claim 10 further comprising an aspirator or air amplifier connecting with the container outlet.

16. A device for destroying ozone, comprising:
a container containing a catalyst and forming a flowpath through the catalyst, with the flowpath having a first segment extending in a first direction towards a first end of the container, and a second segment connecting with the first segment, and with the second segment of the flow path extending in a second direction, through the catalyst and towards a second end of the container.

17. The device of claim 16 wherein the first end of the container is the lower end and the first direction is a vertically downwardly direction, and wherein the second end is an upper end of the container, and wherein the second direction is a vertically upwardly direction, opposite to the first direction.

18. The device of claim 16 further comprising a second container within the first container, the second container having an inlet end and an outlet end, and containing the catalyst in between the inlet end and the outlet end, and with one or more gas inlets at the inlet end, and a gas exhaust line connecting directly or indirectly to the outlet end.

19. A method for destroying ozone, comprising the steps of:
moving the ozone in a first direction within a container;
moving the ozone through a catalyst, in a second direction different from the first direction, and with the second direction having a vertical component, with the catalyst converting at least some of the ozone into oxygen; and
moving the oxygen and any remaining ozone out of the container.

20. The method of claim 19 further comprising the steps of:
collecting any condensate at a lower end of the container and draining it out of the container.
